(19) United States
(12) Plant Patent Application Publication
(10) Pub. No.: US 2016/0249508 P1
(43) Pub. Date: Aug. 25, 2016

(54) Raspberry plant named 'Pacific Starlet'

(50) ;

(71) Applicant: Ellen Thompson, Watsonville, CA (US)

(72) Inventor: Ellen Thompson, Watsonville, CA (US)

(73) Assignee: Pacific Berry Breeding, LLC, Salinas, CA (US)

(21) Appl. No.: 14/544,826

(22) Filed: Feb. 24, 2015

(51) Int. Cl. A01H 5/00

(52) U.S. Cl. PLT/204

(57) ABSTRACT

A new and distinct cultivar of Raspberry plant named ‘Pacific Starlet’ as described and shown herein. ‘Pacific Starlet’ provides very firm berries which detach easily from the receptacle and maintain bright non-darkening color in postharvest cold storage.

### BACKGROUND AND SUMMARY

The new raspberry cultivar designated ‘Pacific Starlet’ is shown herein. Botanically known as Rubus idaeus L., ‘Pacific Starlet’ is a primocane-fruiting raspberry that was discovered in a seedling field in May 2010 in Watsonville, Calif. ‘Pacific Starlet’ originated from open pollinated (OP) seed of an unknown variety. An increasing number of patented primocane-fruiting raspberries are discovered using material sourced from OP fruit. One example is the raspberry cultivar ‘Vintage’ (U.S. Plant Pat. No. 24,198), which was derived from open pollinated seed of the patented proprietary variety ‘Isabel’ (U.S. Plant Pat. No. 9,340). Another example is the variety ‘Diamond Jubilee’ (U.S. Plant application Ser. No. 13/694,890), which was derived from open-pollinated fruit that was purchased in a retail store and thought to be the patented proprietary variety ‘Driscoll Maravilla’ (U.S. Plant Pat. No. 14,804).

The present cultivar, ‘Pacific Starlet’, offers significant advantages over the existing, patented primocane-raspberry cultivar ‘Vintage’ (U.S. Plant Pat. No. 24,198). For example, ‘Pacific Starlet’ offers typically firmer fruit than ‘Vintage’. Another example of an existing patented primocane-raspberry is ‘Marcela’ (U.S. Plant Pat. No. 17,819). In contrast to ‘Marcela’, ‘Pacific Starlet’ has much larger fruit, though both are firm and have bright non-darkening color. A third example of an existing, patented primocane-raspberry variety is ‘Pacific Deluxe’ (U.S. Plant Pat. No. 21,074), a previous release from the same breeding program as the present cultivar, ‘Pacific Starlet’. The present cultivar, ‘Pacific Starlet’ is a significant improvement over ‘Pacific Deluxe’ particularly for color; ‘Pacific Deluxe’ tends to darken significantly in postharvest cold storage, whereas ‘Pacific Starlet’ does not darken when picked and cold stored. Other improvements of ‘Pacific Starlet’ over ‘Pacific Deluxe’ include reduced fruit defects, easier fruit detachment, and lower acidity.

Thus, ‘Pacific Starlet’ is a new and distinct primocane-raspberry cultivar that can be identified, among other things, by its bright non-darkening berry color in cold storage, large size, ease of detachment and lower acidity. Further, leaf hue of ‘Pacific Starlet’ is distinct, with an upper color of RHS 137D often flecked with the yellowish-green color RHS 151D.

### BRIEF DESCRIPTION OF THE PHOTOGRAPHS

FIG. 1 is a photograph showing fruit of the variety Raspberry cultivar ‘Pacific Starlet’ to have an external fruit color of RHS 44A.

FIG. 2 is a photograph showing the canopy of the Raspberry cultivar ‘Pacific Starlet’ having a crown age of about 3.5 years and a cane age of about 8 months.

FIG. 3 is a close-up photograph showing ripe and unripe fruit and leaf detail of the Raspberry cultivar ‘Pacific Starlet’ having a crown age of about 3.5 years and a cane age of about 8 months.

FIG. 4 is a further close-up photograph showing ripe and unripe fruit and leaf detail of the Raspberry cultivar ‘Pacific Starlet’ having a crown age of about 3.5 years and a cane age of about 8 months.

FIG. 5 is a copy of the UPOV technical questionnaire for the Raspberry cultivar ‘Pacific Starlet’.

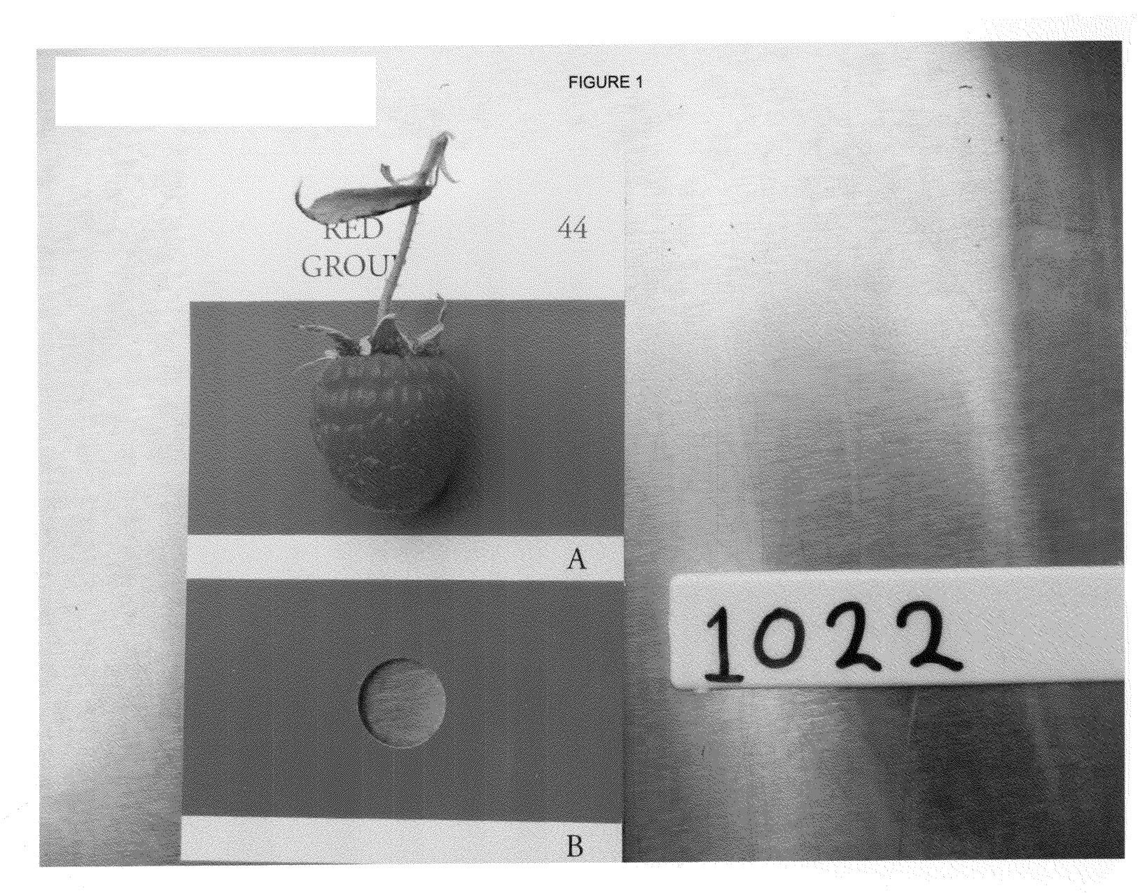

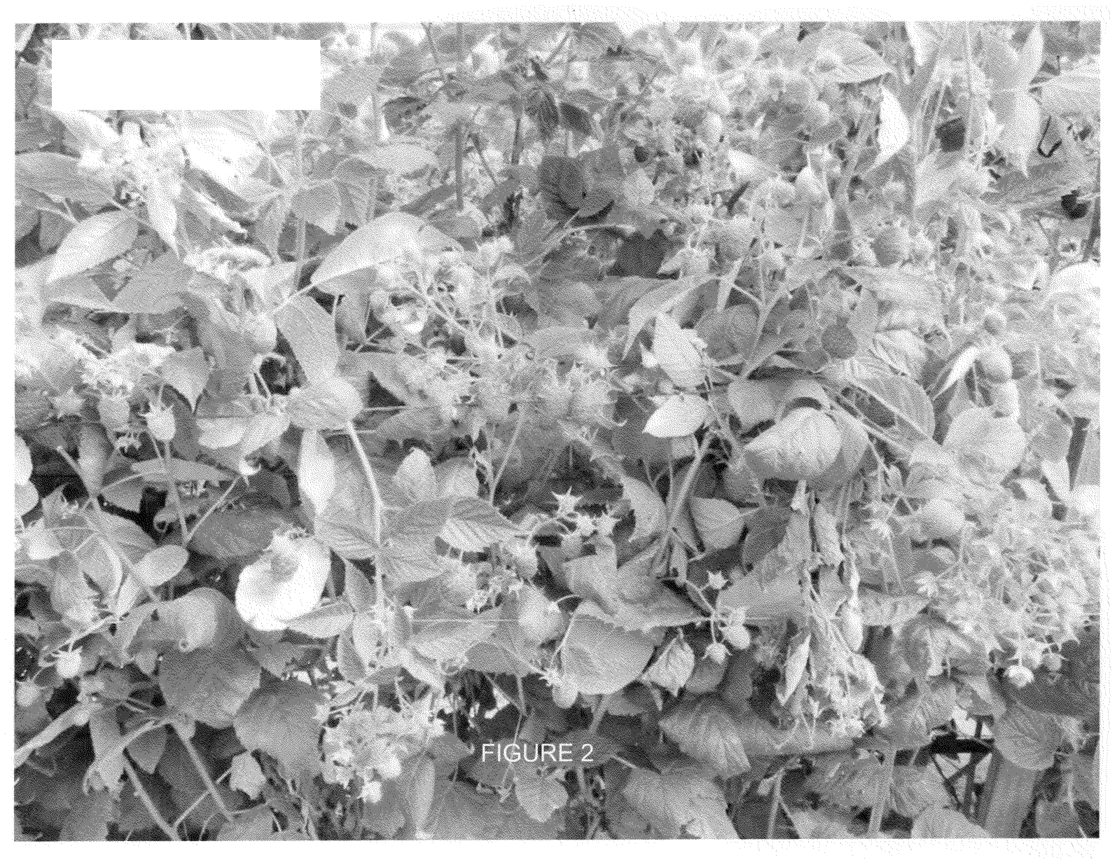

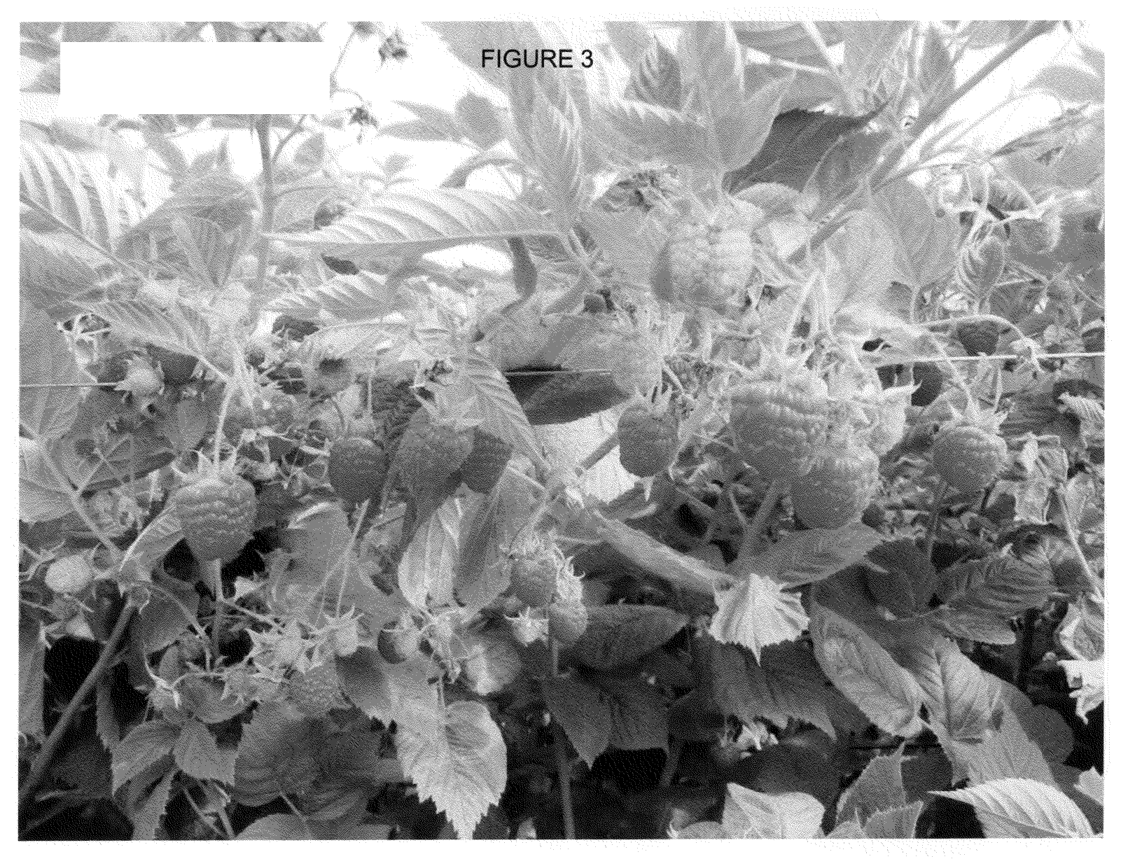

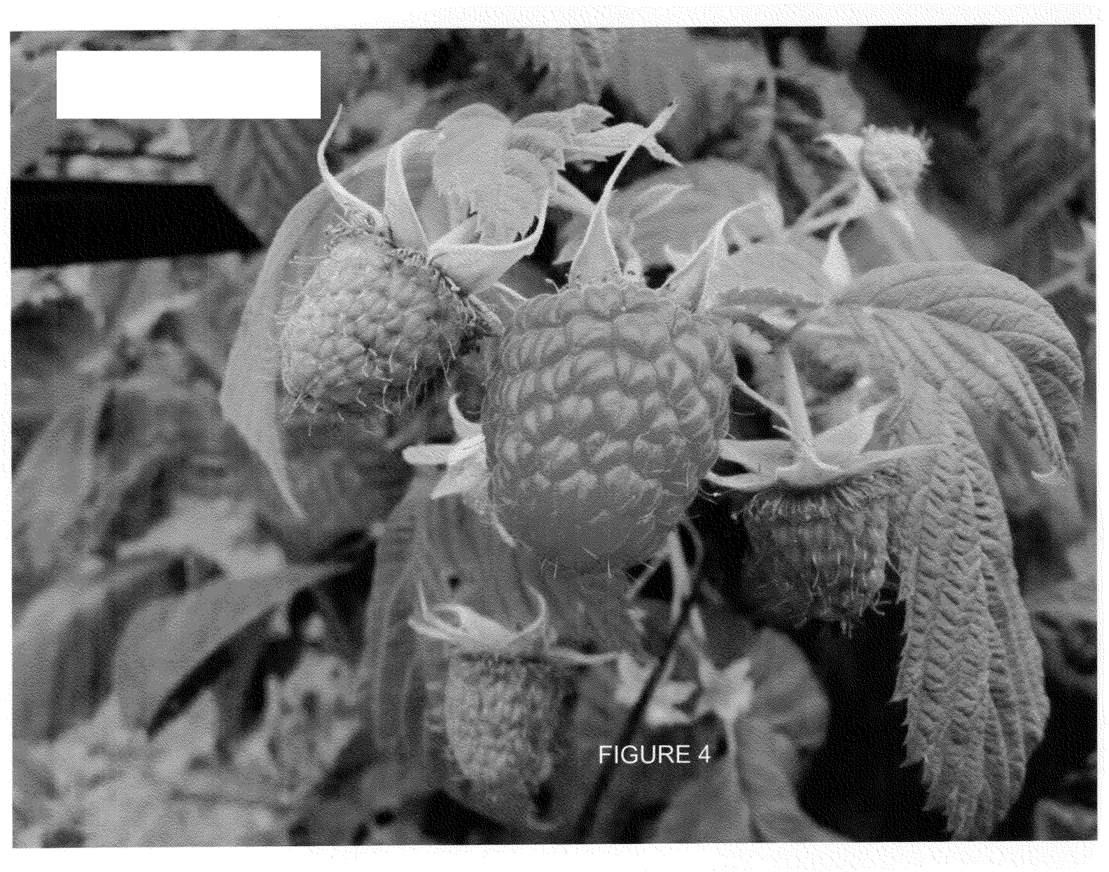

### DETAILED DESCRIPTION

Note: statements of characteristics herein represent exemplary observations of the cultivar herein and will vary depending on time of year, location, annual weather, etc.
Cultivar name: ‘Pacific Starlet’
Classification:
   Family.—Rosaceae.
   Botanical name.—Rubus idaeus.
   Common name.—Raspberry.
Parentage:
   Female parent (please provide name or code).—Unknown.
   Male parent (please provide name or code flower).—Unknown. ‘Pacific Starlet’ was first identified in a field with other seedlings in May 2010 at Watsonville, Calif. USA. ‘Pacific Starlet’ was first propagated asexually by crown division in August 2010 in Watsonville, Calif. USA. The crown on the original plant was dug and parted into basal cane pieces (approximately 15 cm long) with root attached and replanted into a selection plot elsewhere on the farm, resulting in a 3-fold increase. In September 2011, two actively growing primocanes were dug (with root attached) and shipped to Lafayette, Oreg. USA, where vegetative material was explanted and established in vitro for micropropagation.
Growing location for the observations herein: Watsonville, Calif. USA.
Time of year (season): Autumn.
Age of plants used for this discussion: Crown age of about 3.5 years and a cane age of about 8 months.
Age of plants used for the photographs in the figures: Crown age of about 3.5 years and a cane age of about 8 months.
Type of greenhouse covering or growing structure, or field: High tunnel over field.
Light: Natural.
References to color refer to the RHS — Fifth Edition. Observations for floricanes herein were made in June 2013. Observations for primocanes herein were made in August 2013.
Plant:
   Form/shape.—Vase.
   Growth habit.—Erect.
   Height.—1.9 m as measured from cane base to cane apex.
   Spread.—40.8 cm as measured from lateral leaf tip to lateral leaf tip.
   Propagation method.—(stolons, tissue culture, division, tip cuttings): Division.
   Time to initiate and develop roots.—24 days.
   Root description.—Generally of thick diameter with a smooth, glossy texture. Few feeder roots present. Few suckers emerge from the roots. Generally not an aggressive spreader.
Primocanes:
   Diameter.—Base: 1.1 cm|Middle: 0.9 cm|Tip: 0.3 cm.
   Length.—1.5 m.
   Number of nodes.—37.
   Internode length.—Base: 3.4 cm|Middle: 6.5 cm|Tip: 4.0 cm.
   Number of canes/hill.—4-6.
   Cane color.—RHS 144C.
   Spines (present or absent).—Present Density: Base: 28/cm²|Middle: 0.5/cm²|Tip: 2/cm² Shape: Acute Length: 0.05 cm Width: 0.01 cm Apex descriptor: Lanceolate Color: RHS 181A.
   Bud shape.—Rounded Length: 0.3 cm Diameter (base): 0.2 cm Diameter (tip): 0.08 cm Color: RHS N200B.
Floricanes:
   Diameter.—Base: 1.1 cm|Middle: 0.9 cm|Tip: 0.8 cm.
   Length.—1.9 m.
   Number of nodes.—14.
   Internode length.—Base: 7.2 cm|Middle: 9 cm|Tip: 10.8 cm.
   Cane color.—RHS 175A.
   Spines (present or absent).—Present Density: Base: 28/cm²|Middle: 0.5/cm²|Tip: 2/cm² Shape: Acute Length: 0.05 cm Width: 0.01 cm Apex descriptor: Lanceolate Color: RHS 181A.
   Bud shape.—Rounded Length: 0.3 cm Diameter (base): 0.2 cm Diameter (tip): 0.08 cm Color: RHS N200B.
Leaves:
   Complete leaf.—Length: 19.8 Width: 17.4 Number of leaflets: 5.
   Terminal leaflet.—Size: Length (cm): 9.6 cm Width (cm): 7.2 cm Length/Width ratio: 1.33 Shape of apex: Acuminate Shape of Base: Terminal — Cordate Basal — Sessile Margin: Serrate Texture: Mild interveinal puckering Number of serrations/leaf: 96 Shape of serrations: Flexuous — Concave Color: Upper Surface: RHS 137A Lower Surface: RHS 188C Venation pattern: Pinnate Venation Color: Upper surface: RHS N144D Lower surface: RHS 145B Leaf pubescence density: None, glabrous Color of leaf pubescence: N/A Shape of leaf in cross-section: Number of leaflets/leaf: 3 - 5 Interveinal blistering: Mild Glossiness: Low.
   Primocane leaves.—Petiole length: 4.1 cm Petiole diameter: 2.0 cm Petiole Color: Upper: RHS 137D Lower: RHS 143D Rachis length: 2.0 cm Stipule length: 0.6 cm Stipules per leaf: 2 Stipule Width: 0.01 cm Stipule Color: RHS N144D Color: Upper Surface: RHS 137A Lower Surface: RHS 188C Terminal leaflet: Length: 9.6 cm Width: 7.2 cm Rachis length: 0.8 cm Basal lateral leaflet: Length: 7.2 cm Width: 4.3 cm Petiolule length: 0.1 cm.
   Floricane leaves.—Petiole length: 4.8 cm Stipule length: 0.4 cm Stipules per leaf: 2 Stipule Width: 0.01 cm Stipule Color: RHS N144D Color: Upper surface: RHS 137A Lower surface: RHS 188C.
   Terminal leaflet.—Length: 9.0 cm Width: 6.0 cm Rachis length: 1.7 cm Distal lateral leaflet: Length: 8.4 cm Width: 6.0 cm Petiolule: Length: 1.7 cm Diameter: 0.1 cm Color: Upper — RHS 137D Lower — RHS 143D.
   Basal lateral leaflet.—Length: 7.2 cm Width: 4.2 cm Petiolule Length: 0.1 cm Diameter: 0.1 cm Color: Upper — RHS 137D Lower — RHS 143D.
Flowers:
   Time of flowering (50% of plants at first flower).—August 5 on primocanes.
   Size.—Length: 0.9 cm Diameter: 0.7 cm.
   Fragrance.—None.
   Peduncle.—Length: 0.8 cm Diameter: 0.05 cm Color: RHS 143B Pubescence: Present Texture: Smooth with few undulations.
   Perianth.—Flowering trusses shape: Truncate.
   Petals.—Color (upper and lower): RHS 149D Number per flower: 5 Shape: Oblanceolate Length: 0.6 cm Width: 0.1 cm Apex descriptor: Rounded Base Descriptor: Truncate Margin descriptor: Entire.
   Sepals.—Quantity: 5 Length: 0.6 cm Width: Base — 0.3 cm Mid — 0.1 cm Tip — 0.01 cm Color: RHS 145B Apex descriptor: Acuminate Margin descriptor. Entire.
   Pedicel.—Color: RHS 145A Length: 0.9 cm Diameter: 0.01 cm.
Reproductive organs:
   Self-fertile.—yes/no.
   Male.—Stamen Number: 90 Filament Length: 0.3 cm Diameter: 0.01 cm Color: RHS 155C Anther Length: 0.01 cm Diameter: 0.01 cm Color: RHS 161C Pollen Color: RHS 161C Amount: (none, sparse, moderate, heavy).
   Female.—Style Length: 0.2 cm Diameter: 0.01 cm Color: RHS 154D Stigma Length: 0.2 cm Diameter: 0.01 cm Color: RHS 154D.
Fruit:
   Predominant shape.—Broad conic.
   Weight (g).—4.2.
   Length.—2.1 cm.
   Width.—1.75 cm.
   Length/width ratio.—1.2.
   Receptacle length.—1.8 cm.
   Diameter.—Base — 0.7 cm Middle — 0.65 cm Tip — 0.2 cm.
   Color.—RHS 143B.
   Drupelet length.—0.6 cm.
   Diameter.—0.4 cm.
   Number.—80.
   Weight.—0.3 g.
   Fruit color external.—RHS 44A.
   Internal.—RHS 46A.
   Firmness of skin.—Very firm.
   Firmness of flesh.—Very firm.
   Hollow center.—Present.
   Number of fruit per node.—4-6.
   Time of ripening (50% of plants with first fruit).—September 10 on primocanes.
   Time of fruiting.—Early summer and autumn.
   Type of bearing.—Remontant.
   Fruit yield.—15, 750 lb/a.
   Average brix°.—8.2.
   Market use.—Fresh.
   Keeping quality.—Excellent.
   Shipping quality.—Excellent.
   Typical market use.—Fresh.
   Keeping quality.—Very good.
   Shipping quality.—Excellent.
Pest and disease resistance: Pacific Starlet exhibits resistance to Raspberry Bushy Dwarf Virus (RBDV). Susceptibility to yellow rust (Phragmidium) has been observed. Exhibits moderate field tolerance to Phytopthora root rot.

What is claimed is:

1. A new and distinct cultivar of Raspberry plant named ‘Pacific Starlet’ as described and shown herein.
